(19) United States
(12) Patent Application Publication
(10) Pub. No.: US 2011/0165279 A1
(43) Pub. Date: Jul. 7, 2011

(54) SWEET POTATO TRYPSIN INHIBITOR AND METHODS FOR TREATING INFLAMMATION AND HYPERALGESIA

(75) Inventors: Yaw-Huei LIN, Taipei (TW); Guan-Jhong HUANG, Taipei (TW)

(73) Assignee: Academia Sinica Office of Public Affairs (Technology Licensing), Taipei (TW)

(21) Appl. No.: 12/985,960

(22) Filed: Jan. 6, 2011

Related U.S. Application Data
(60) Provisional application No. 61/292,579, filed on Jan. 6, 2010.

(51) Int. Cl. A61K 36/00; A61P 29/00

(52) U.S. Cl. 424/773; 424/774

(57) ABSTRACT

Some embodiments of the invention are directed to a pharmaceutical composition comprising an anti-inflammatory or an anti-hyperalgesic effective amount of isolated or purified Ipomomoea batatas trypsin inhibitor and a pharmaceutically acceptable carrier. The Ipomomoea batatas trypsin inhibitor may be derived from a sweet potato leaf or storage root. Also contemplated are methods of treating inflammation or hyperalgesia comprising selecting a subject suffering from inflammation or hyperalgesia, such as a subject with edema; and administering an anti-inflammatory or anti-hyperalgesia effective amount of amount Ipomomoea batatas trypsin inhibitor or a composition comprising an amount of Ipomomoea batatas trypsin inhibitor to the subject; wherein inflammation or hyperalgesia in the subject is reduced upon administration.

This application claims priority to U.S. Provisional Application No. 61/292,579 filed Jan. 6, 2010, which is incorporated herein by reference in its entirety.

### BACKGROUND

1. Field of the Invention

Trypsin inhibitor (IbTI) is found in the leaves and storage roots of sweet potato (Ipomoea batatas (L.) Lam. ‘Tainong 57’) with the activity of anti-inflammatory and anti-hyperalgesic effects using lipopolysaccharide (LPS)-stimulated mouse macrophage cell line (RAW 264.7) and carrageenan-induced mouse paw edema model in vivo, respectively.

2. Description of the Related Art

Two kinds of pain have been described in literature: inflammatory pain and neuropathological pain. Inflammatory stimuli including pathogens, non-self molecules (e.g., endotoxin lipopolysaccharide), aged or damaged self molecules induce cytokines, which mediate tissue responses in different phases of inflammation in a sequential and concerted manner and may result in activation of innate immune system, disseminated intravascular coagulation, multiple organ failure, shock and even death [Rainsford, K. D., Subcell. Biochem. 2007, 42, 3-27]. Macrophage activation by bacterial LPS promotes the secretion of proinflammatory cytokines, such as tumor necrosis factor-α (TNF-α) and interleukin-1β (IL-1β). These substances are important regulators of both innate and adaptive immunity. Moreover, LPS induces inducible nitric oxide synthase (iNOS) and cyclooxygenase-2 (COX-2) gene expression in rat liver, and the COX enzyme possesses both cyclooxygenase and peroxidase functions [Ohshima et al., Mutat. Res. 2005, 591(1-2), 110-122].

COX-2 is expressed at very low levels and is strongly induced by growth factors and several activated oncogenes [Hofseth L. J. & Wargovich, M. J., J. Nutr. 2007, 137, 183-185]. The significance of COX-2 in prostaglandin synthesis and inflammation is highlighted by the observation that COX-2 inhibitors block the synthesis of PGE₂, and as a result, they inhibit inflammation and confer analgesia [Jachak S M. Curr. Opin. Investig. Drugs. 2007, 8(5), 411-415].

Nitric oxide (NO) has been identified as a neurotransmitter in the central nervous system and a potent vasorelaxant that physiologically regulates blood pressure through modulating muscular tone [Hibbs et al., Science 1987, 235, 473-476]. NO also has been defined as an important molecule in inflammation and sepsis [Wheeler, A. P. & Bernard, G. R., New Engl. J. Med. 1999, 340, 207-214]. NO is produced by nitric oxide synthase (NOS), a family of enzymes composed of three isoforms. Neuronal NOS (nNOS, type I) and endothelial NOS (eNOS, type III) are Ca²⁺- and calmodulin-dependent constitutive isoforms; while the calcium-independent isoform (iNOS, type II) is inducible. nNOS has a function in neurotransmission; eNOS plays an important role in vasorelaxation, and the NO produced by the endothelium has antithrombotic properties. After exposure to endogenous and exogenous stimulators, iNOS can be induced in various cells such as macrophages, smooth muscle cells, and hepatocytes to trigger several disadvantageous cellular responses, as well as causing some diseases including inflammation, sepsis, and stroke [Duval et al., Mol. Pharmacol. 1996, 50, 277-284; Rockey, D. C. & Chung, J. J., Am. J. Physiol. 1996, 271, 260-267]. Therefore, NO production induced by iNOS may reflect the degree of inflammation and provide a measure to assess the effect of drugs on the inflammatory process. Recently, it was demonstrated that inhibitors of iNOS might offer some protection in LPS-induced hepatic toxicity and that natural antioxidants such as curcumin, resveratrol, and tea polyphenols exhibit inhibitory effects on LPS-induced iNOS and hepatic damage [Brouet et al., Biochem. Biophys. Res. Commun. 1995, 206, 535-540; Tsai et al., Br. J. Pharmacol. 1999, 126, 673-680; Pan et al., Biochem. Pharmacol. 2000, 59, 357-367; Zhang, C. et al., J. Pharmacol. Exp. Ther. 2000, 293, 968-972].

Native serine proteinase inhibitors usually behave as pseudo-substrates and the amino acid at position P1 drives the inhibitory specificity [Bode, W. & Huber, R. Structural basis of the proteinase-protein inhibitor interaction. In: F. X. Avile's (Ed.), Innovations in Proteases and Their Inhibitors, Springer, Berlin, 1993, pp. 81-122; Macbride et al., J. Mol. Biol. 1996, 259, 819-827]. Serine proteinase inhibitors act against inflammation. Plant Kunitz trypsin inhibitors (KTI) from soybean could inhibit the up-regulation of urokinase expression through suppression of the mitogen-activated protein kinase (MAPK)-dependent signaling [Kobayashi et al., J. Periodontal Res. 2005, 40(6), 461-468]. KTI may induce suppression of proinflammatory responses through suppression of some signaling cascades.

Sohonie and Bhandarker first reported the presence of TI in sweet potato (IbTI) [J. Sci. Ind. Res., 1954, 13B, 500-503]. IbTI accounted for about 60% of total water-soluble proteins in sweet potato roots and could be considered as storage proteins [Lin et al., Bot. Bull. Acad. Sin., 1980, 21, 1-13]. Maeshima et al. identified sporamin as the major storage protein in sweet potato root, which accounted for 80% of total proteins in root [Phytochemistry, 1985, 24, 1899-1902]. Lin proposed that sporamin should be one form of TI in sweet potato, which was confirmed later [Lin Y H. Trypsin inhibitors of sweet potato: review and prospect, in: Y. I. Hsing, C. H. Chou, (Eds.), Recent Advances in Botany, Academia Sinica Monograph series, No. 13, Taipei, Taiwan, 1993, pp. 179-185; Yeh et al., Plant Mol. Biol. 1997, 33, 565-570; Caj et al., Plant Mol. Biol. 2003, 51, 839-849]. IbTI exhibited dehydroascorbate reductase, monodehydroascorbate reductase and antioxidant activities and also angiotensin converting enzyme inhibitory activity [Hou et al., J. Agric. Food Chem. 2001, 49, 2978-2981; Huang et al., Botanical Studies, 2008, 49, 101-108].

Injection of aprotinin, a serine-type proteinase inhibitor from bovine lung, was shown by Digiesi et al. to increase the initial pain limit walking tolerance and to decrease the intensity of pain at rest and of myalgic or trigger areas in patients with peripheral arterial disease [Digiesi et al., Pain. 1975, 1, 385-389]. The authors proposed that aprotinin might inhibit kininogenases and tissue protein-hydrolyzing enzymes activated in the course of ischemia; however, they did not show the details. Shih et al. showed that pre- or post-treatment with C-phycocyanin, a biliprotein from blue green algae, (30 or 50 mg/kg, IP) significantly attenuated carrageenan-induced thermal hyperalgesia and the induction of iNOS and COX2, accompanied by an inhibition of the formation of TNF-alpha, prostaglandin E(2), and neutrophil infiltration into inflammatory sites [Anesth. Analg. 2009, 108, 1303-1310].

### SUMMARY

Some embodiments of the invention are directed to a pharmaceutical composition comprising an anti-inflammatory or an anti-hyperalgesic effective amount of isolated or purified Ipomomoea batatas trypsin inhibitor and a pharmaceutically acceptable carrier. The Ipomomoea batatas trypsin inhibitor may be derived from a sweet potato leaf or storage root.

Also contemplated are methods of treating inflammation or hyperalgesia comprising selecting a subject suffering from inflammation or hyperalgesia, such as a subject with edema; and administering an anti-inflammatory or anti-hyperalgesia effective amount of Ipomomoea batatas trypsin inhibitor or a composition comprising Ipomomoea batatas trypsin inhibitor to the subject; wherein inflammation or hyperalgesia in the subject is reduced upon administration. The Ipomomoea batatas trypsin inhibitor or a composition comprising Ipomomoea batatas trypsin inhibitor may inhibit iNOS or COX-2 production, TNF-α production, NO production, protein kinase C (PKC) production and/or PGE2 production upon administration.

### BRIEF DESCRIPTION OF THE DRAWINGS

FIG. 1. Effects of sweet potato trypsin inhibitor (IbTI) on lipopolysaccharide (LPS)-induced cell viability (A) and NO production (B) of RAW 264.7 macrophages. Cells were incubated for 24 hours with 1 μg/ml of LPS in the absence or presence of IbTI (0, 5, 10, 20, 40 μM) (0, 125, 250, 500, 1,000 μg/mL). IbTI was added 1 hour before incubation with LPS. Cell viability assay was performed using MTT assay. Nitrite concentration in the medium was determined using Griess reagent. The data were presented as mean±S.D. for three different experiments performed in triplicate. # compared with sample of control group. *p<0.05 and **p<0.01 were compared with LPS-alone group.

FIG. 2. Effects of sweet potato trypsin inhibitor on TNF-α (FIG. 2A) and PGE2 (FIG. 2B) production by LPS-induced RAW 264.7 macrophages. Cells were incubated for 24 hours with 1 μg/ml of LPS in the absence or presence of IbTI (0, 5, 10, 20, and 40 μM) (0, 125, 250, 500, and 1,000 μg/mL). IbTI was added 1 hour before incubation with LPS. The culture medium was collected for assay with TNF-α and PGE2 ELISA kit. The data were presented as mean±S.D. for three different experiments performed in triplicate. # compared with sample of control group. **p<0.01 was compared with LPS-alone group.

FIG. 3. Inhibition of iNOS and COX-2 protein expression by sweet potato trypsin inhibitor in LPS-stimulated RAW264.7 cells. Cells were incubated for 24 hours with 1 μg/ml of LPS in the absence or the presence of IbTI (0, 5, 10, 20, and 40 μM) (0, 125, 250, 500, and 1,000 μg/mL). IbTI was added 1 hour before incubation with LPS. Lysed cells were then prepared and subjected to western blotting using an antibody specific for iNOS and COX-2. β-actin was used as an internal control. (A) A representative western blot from two separate experiments is shown. (B) Relative iNOS and COX-2 protein levels were calculated with reference to a LPS-stimulated culture. # compared with sample of control group. The data were presented as mean±S.D. for three different experiments performed in triplicate. **p<0.01 and ***p<0.001 were compared with LPS-alone group.

FIG. 4. Inhibition of MMP-9 and MMP-2 protein expression by sweet potato trypsin inhibitor in LPS-stimulated RAW264.7 cells. Cells were pretreated with IbTI (0, 10, 20, and 40 μM) (0, 250, 500, and 1,000 μg/mL) for 1 hour before being incubated with LPS (1 μg/mL) for 24 h. (A) Cells suspended were then prepared and the MMP-9 and MMP-2 activity were detected using SDS-PAGE zymography. (B) Relative MMP-9 and MMP-2 protein levels were calculated with reference to a LPS-stimulated control culture. (C) Inhibition of MMP-9 protein expression by IbTI in LPS-stimulated RAW264.7 cells using an antibody specific for MMP-9. β-actin was used as an internal control. (D) Relative MMP-9 protein level was calculated with reference to a LPS-stimulated control culture. The data were presented as mean±S.D. for three different experiments performed in triplicate. ***p<0.001 was compared with positive control group.

FIG. 5. Effects of sweet potato trypsin inhibitor and indomethacin (Indo) on hind paw edema induced by λ-carrageenan in mice. The data were presented as mean±S.D. for three different experiments performed in triplicate. **p<0.01 and ***p<0.001 were compared with the λ-carrageenan (Carr) group.

FIG. 6. Effects of sweet potato trypsin inhibitor and indomethacin (Indo) on the MDA concentration of mice paws. Each value represents mean±S.D. ###p<0.001 as compared with the control group. **p<0.01 and ***p<0.001 were compared with the λ-carrageenan (Carr) group.

FIG. 7. Effects of IbTI and indomethacin (Indo) on carrageenan (Carr)-induced TNF-α (A) and NO (B) concentration of serum at 5th hour in mice. The data were presented as mean±S.D. for three different experiments performed in triplicate. ###p<0.001 as compared with the control group. *p<0.05, **p<0.01 and ***p<0.001 were compared with the λ-carrageenan (Carr) group.

FIG. 8. Histological appearance of the mouse hind footpad after a subcutaneous injection with 0.9% saline (Control group) or carrageenan (Carr) stained with hematoxylin and eosin. (A) Control rats: show the normal appearance of dermis and subdermis without any significantly lesion. (B) Hemorrhage with moderately extravascular red blood cell and large amount of inflammatory leukocyte mainly neutrophils infiltration in the subdermis interstitial tissue of mice following the subcutaneous injection of Carr only. Moreover, detail of the subdermis layer show enlargement of the interstitial space caused by edema with exudates fluid. (C) Indomethacin (Indo) significantly reduced the level of hemorrhage, edema and inflammatory cell infiltration compared to subcutaneous injection of Carr only. (D) IbTI (40 mg/kg) significantly show morphological alterations compared to subcutaneous injection of Carr only. (100×.) (E) The numbers of neutrophils were counted in each scope (400×) and their average counts from 5 scopes of every tissue slice were calculated. **P<0.01, compared with Carr group.

FIG. 9. Effects of sweet potato trypsin inhibitor (IbTI) and indomethacin (Indo) on MMP-9 and MMP-2 expression induced by λ-carrageenan in mice. (A) Tissue suspended was prepared and the MMP-9 and MMP-2 activity were detected using SDS-PAGE zymography. (B) Relative MMP-9 and MMP-2 protein levels were calculated with reference to blank. The data were presented as mean±S.D. for three different experiments performed in triplicate. **p<0.01 and ***p<0.001 were compared with the λ-carrageenan (Carr) group.

### DETAILED DESCRIPTION

Trypsin inhibitor from sweet potato (Ipomoea batatas (L.) Lam. ‘Tainong 57’) leaves and storage roots (IbTI) have been found to have anti-inflammatory and anti-hyperalgesic effects.

The Examples shown below highlight the versatility of trypsin inhibitor, and show some of the antioxidant and anti-inflammatory mechanisms of IbTI using both mouse macrophage cell line (RAW 264.7) ex vivo and Carr-induced paw edema in the ICR mouse.

Since hydrogen peroxide is a novel mediator of inflammatory hyperalgesia, acting via transient receptor potential vanilloid 1-dependent and independent mechanisms, although not bound by this theory, our results suggest that the anti-inflammatory effects of IbTI at least in part depend on an anti-free radical mechanism, and on the inhibition of TNF-α, iNOS, COX-2, MMP-9, and possible PKC, expression at the protein level [Ferreira et al., Pain. 2005, 117,171-181; Keeble et al., Pain. 2009, 141, 135-142]. The inhibition of MMP-9 at the enzyme activity level is also quite possible.

One aspect of IbTI is the extraction and purification of storage protein forms of both sweet potato leaves and storage roots which is one of the most important root crops in the world. Many drugs have unwanted side effects. One nutritionally based strategy for reducing pain is that of Rivat et al. in which polyamine is eliminated from the diet [Rivat et al., Pain. 2008,137,125-137]. These types of approaches suggest that, with the appropriate knowledge, patients can achieve relief from symptoms with highly specialized and innovative manipulation of dietary intake. Similarly, extraction and purification of the active ingredients from food, and administration to patients, will benefit a huge population of people suffering from inflammation and/or pain.

NO plays a role as neurotransmitter, vasodilator, and immune regulator in a variety of tissues at physiological concentration [Moncada et al., Pharmacol. Rev. 1991, 43, 109-142 (1991)]. High levels of NO produced by iNOS have been defined as a cytotoxic molecule in inflammation [Kroncke et al., Nitric Oxide. 1997, 1, 107-120]. PGE2 is a pleiotropic mediator produced at inflammatory sites by COX-2 and gives rise to pain, swelling, and stiffness [Seibert et al., Proc. Natl. Acad. Sci. USA. 1994, 91, 12013-12017]. Thus, potential inhibitors of iNOS and COX-2 have been considered to be potential anti-inflammatory drugs

As discussed below in Examples 1-4 these effects were measured using lipopolysaccharide (LPS)-stimulated mouse macrophage cell line (RAW 264.7) in vitro. The inhibitory effects of IbTI were also shown in the carrageenan-induced mouse paw edema model in vivo (Example 5). When RAW 264.7 macrophages were treated with different concentrations of IbTI (0, 125, 250, 500, and 1,000 μg/mL, corresponding to 0, 5, 10, 20, and 40 μM respectively) together with LPS (1 μg/mL), a significant concentration-dependent inhibition of nitrite production was detected (See Examples 1 and 3 below). As seen in Example 2, LPS-induced TNF-α and PGE2 production was inhibited by pretreatment with IbTI in a dose dependent manner. Western blotting revealed that IbTI decreased both LPS-induced iNOS and COX-2 expression at the protein level by 77.8% and 91.6%, respectively (Example 3). LPS-induced MMP-9 production was inhibited by pretreatment with IbTI in a dose dependent manner demonstrated by SDS-zymography (Example 4). Western blotting showed an average of 40.1% and 48.9% down-regulation of MMP-9 protein expression, respectively, after treatment with IbTI at 500 and 1,000 μg/mL compared with the LPS-alone.

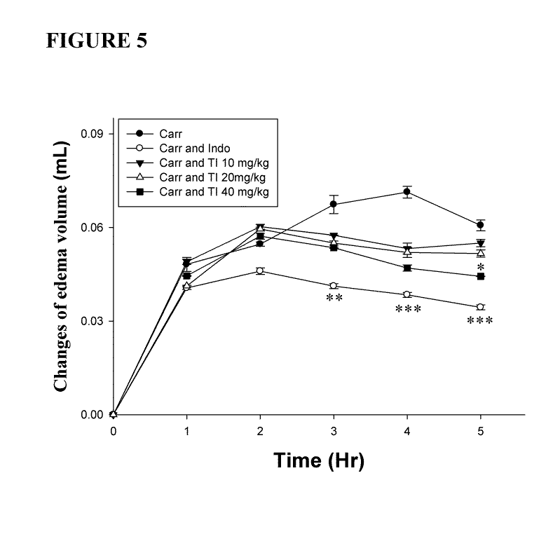

Anti-hyperalgesic effects of IbTI (0, 10, 20, and 40 mg/kg body weight; i.p.) were also shown in Example 5 in the λ-carrageenan (Carr)-induced mouse paw edema model. As can be seen in FIG. 5, treatment with IbTI significantly inhibited edema caused by Carr injection. Malondialdehyde (MDA) is a reactive species that is an indicator of oxidative stress. MDA is also a thiobarbituric reactive substance (TBARS). We found blockage of the thiobarbituric acid reactive substances (TBARS) formation and of MMP-9 expression in treated paws and inhibition of serum nitric oxide (NO) and tumor necrosis factor (TNF)-alpha production by IbTI; i.p. treatment with IbTI also diminished neutrophil infiltration into sites of inflammation as did indomethacin (Examples 6-9). We also outline a method of extracting and purifying IbTI from sweet potato leaf and storage root and producing it in a form that can be conveniently stored for later use.

In some aspects, IbTI exerts its anti-hyperalgesic effects via proteinase inhibitory activity, antioxidant capacities, and suppression of iNOS and COX2 induction and attenuation of TNF-alpha formation.

The terms “approximately, “about,” and “substantially” as used herein represent an amount close to the stated amount that still performs the desired function or achieves the desired result. For example, the terms “approximately,” “about” and “substantially” may refer to an amount that is within less than 10% of, within less than 5% of, within less than 1% of, within less than 0.1% of, and within less than 0.01% of the stated amount.

The term “composition” refers to a mixture of IbTI with other components, such as diluents or additional carriers. The composition facilitates administration of the IbTI to an organism. Multiple techniques of administering a composition exist in the art including, but not limited to, oral, injection, aerosol, parenteral, and topical administration.

The term “pharmaceutically acceptable carrier” refers to a substance or diluent that does not significantly abrogate the biological activity and properties of IbTI. The term “carrier” refers to a chemical compound that facilitates the incorporation of a compound into cells or tissues

The composition may comprise one or more physiologically acceptable surface active agents, additional carriers, diluents, excipients, smoothing agents, suspension agents, film forming substances, and coating assistants, or a combination thereof. Acceptable additional carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, microcrystalline cellulose, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For some embodiments of the composition, a suitable carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

The compositions may, if desired, be presented in a pack or dispenser device which may contain one or more unit dosage forms containing IbTI. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

In some embodiments, in the pharmaceutical industry, it is standard practice to provide substantially pure material when formulating pharmaceutical compositions. Therefore, in some embodiments, “substantially pure” refers to the amount of purity required for formulating pharmaceuticals, which may include, for example, a small amount of other material that will not affect the suitability for pharmaceutical use. In some embodiments, the substantially pure compound contains at least about 96% of the compound by weight, such as at least about 97%, 98%, 99%, or 100% of IbTI.

The term “diluent” refers to chemical compounds diluted in water that will dissolve the composition of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound. As used herein, an “excipient” refers to an inert substance that is added to a composition to provide, without limitation, bulk, consistency, stability, binding ability, lubrication, disintegrating ability, etc., to the composition. A “diluent” is a type of excipient.

The term “inflammation” refers to the body's response to irritants and harmful stimuli. This response may involve local accumulation of fluid, plasma proteins, and white blood cells initiated by physical injury, infection, or a local immune response. Inflammation may be acute and occurring soon after an injury or initiation of an immune response. Chronic inflammation may occur over a prolonged period and may be associated with autoimmune diseases, allergies, harmful irritants or chronic infection. Inflammation may include production of proteins that affect the function of other cells, tissues, organs, and infectious agents. These proteins include, but are not limited to, cytokines, lymphokines, interleukins, chemokines, complement, antibodies, and histamines. Inflammation may also be associated with chemical entities such as nitric oxide, COX-2 activity, prostaglandin synthesis, cyclooxygenase, peroxidase among others. The inflammatory condition may involve phagocytes, dendritic cells, mast cells, basophils, lymphocytes, T-cells, B-cells, natural-killer cells, neutrophils, and eosinophils. The subject may experience pain, the sensation of heat, redness and swelling of tissue, and loss of function.

The term “anti-inflammatory” refers to the inhibition, interference, modulation, or reduction, of mediators of inflammation. There are many possible mechanisms involved in inhibiting inflammation as well as many possible manifestations of anti-inflammatory activity. A sign of anti-inflammatory effect, for example, could be a reduction of iNOS and COX-2 protein expression. Similarly, MMP-9 or MMP-2 protein expression may be depressed compared to the untreated disease state. An anti-inflammatory effect may be observed by the reduction in reactive oxygen species or free radicals as well as a reduced oxidation of lipids. Clinical manifestations of successful treatment with IbTI may include a heightened threshold for pain or a reduction of pain severity or duration in the subject. One of skill in the art would realize that there are many indicators of successful anti-inflammatory treatment.

The term “hyperalgesia” refers to a heightened sensitivity to pain which may be the result of physical or psychological disorder, or disease progression. Pain may be an unpleasant sensory and/or emotional experience arising from actual or potential tissue damage. The pain may be accompanied by anxiety, diaphoresis, nausea, and vital sign changes. Chronic pain is persistent pain that lasts beyond the normal healing period. Hyperalgesia may be localized to discrete areas of the subject and may encompass the entire body. Localized pain may be associated with injury resulting in sensitivity at the site of tissue damage or sensitivity in the surrounding undamaged tissue. Hyperalgesia may be induced by inflammation, tissue injury, allergies, autoimmunity and other disease processes.

The term “anti-herperalgesic” refers to inhibition, interference, reduction, or modulation of the mediators and experience of pain. A successful anti-hyperalgesic treatment may manifest in the subject as modifying the character, onset, location, and duration of the painful symptoms. The treatment may reduce factors that exacerbate pain or promote factors that relieve pain. The anti-herperalgesic treatment may operate in the same manner as described for the modification of inflammation as described above or by some other mechanism.

The term “effective amount” refers to an amount achieving a desired result. In some embodiments, an effective amount of IbTI is an amount that ameliorates inflammation and/or hyperalgesia, for example, in a mammalian subject (e.g., a human). The effective amount of IbTI disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, an effective amount means an amount of IbTI effective to alleviate or ameliorate inflammation and/or hyperalgesia in the subject being treated, or prevent symptoms in a subject suspected of developing inflammation or hyperalgesia. Determining an effective amount and identifying subjects suspected of developing inflammation or hyperalgesia are well within the capability of those skilled in the art. As will be readily apparent to one skilled in the art, the useful in vivo dosage to be administered and the particular mode of administration will vary depending upon the age, weight and mammalian species treated and the specific use for which IbTI is employed. The determination of effective dosage levels, that is the dosage levels necessary to achieve the desired result, can be accomplished by one skilled in the art using routine pharmacological methods. Typically, human clinical applications are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved adverse side effects disappear. The dosage may range broadly, depending upon the desired effects and the therapeutic indication. Dosages may be about 10 microgram/kg to about 100 mg/kg body weight. Alternatively dosages may be based and calculated upon the surface area of the patient, as understood by those of skill in the art.

The exact formulation, route of administration, and dosage for IbTI can be chosen by the individual physician in view of the patient's condition. The dose range of the composition administered to a patient may be from about 0.5 to about 1000 mg/kg of the patient's body weight. The dosage may be a single one or a series of two or more given in the course of one or more days, as is needed by the patient. In instances where human dosages of IbTI have been established for anti-inflammatory or anti-hyperalgesic treatment, the treatment may include dosages that are about 0.1% to about 500% of the established human dosage.

As will be understood by those of skill in the art, in certain situations it may be necessary to administer IbTI in amounts that exceed, or even far exceed a preferred dosage range in order to effectively and aggressively treat particularly aggressive disorders or conditions. In some embodiments, the compounds will be administered for a period of continuous therapy, for example for a week or more, or for months or years.

Used herein, the term “carrier” refers to a chemical compound that facilitates the incorporation of a compound into cells or tissues.

“Derived from” refers to the identity and/or original source of IbTI. The IbTI was originally purified from sweet potato. However, “derived from” also refers to IbTI made or purified from sources other than sweet potato that yield substantially similar trypsin inhibitors. For example, “derived from IbTI” also includes IbTI made by synthetic means. The IbTI compositions may be manufactured in a manner that is itself known, e.g., by means of conventional mixing, dissolving, granulating, dragee-making, levigating, emulsifying, encapsulating, entrapping or tabletting processes or by some as yet unreported method.

IbTI is a versatile protein, having two pairs of disulfide bonds (Cys81-Cys128; Cys177-Cys184), its trypsin inhibitory activity is maximal when two pairs of disulfide bonds are intact. Partial trypsin inhibitory activity remains while either disulfide bond exist.

“Sweet potato storage root” refers to Ipomoea batatas from the family Convolvulaceae. The storage root is a tuberous root which is enlarged to function as a storage organ in the plant.

The term “treat” refers to administering IbTI to a subject per se, or in compositions where IbTI is mixed with other active ingredient(s), as in combination therapy, or suitable carriers or excipient(s). IbTI administration includes oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. IbTI administration includes sustained or controlled release dosage forms, including depot injections, osmotic pumps, pills, transdermal (including electrotransport) patches, and the like, for prolonged and/or timed, pulsed administration at a predetermined rate.

It should be noted that the attending physician would know how to and when to terminate, interrupt, or adjust treatment due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

Although the exact dosage will be determined on an individual basis, in most cases, some generalizations regarding the dosage can be made. The daily dosage regimen for an adult human patient may be, for example, an oral dose of about 0.1 mg to 2000 mg of the IbTI, or about 1 mg to about 500 mg, e.g. 5 to 200 mg. In other embodiments, an intravenous, subcutaneous, or intramuscular dose of IbTI of about 0.01 mg to about 100 mg, or about 0.1 mg to about 60 mg, e.g. about 1 to about 40 mg being used. In some embodiments, the composition is administered 1 to 4 times per day. Alternatively the compositions of the invention may be administered by continuous intravenous infusion, preferably at a dose of up to about 1000 mg per day.

Dosage amount and interval may be adjusted individually to provide plasma levels of the IbTI which are sufficient to maintain the anti-inflammatory or anti-hyperalgesic effect, or minimal effective concentration (MEC). The MEC will vary for each person but can be estimated from in vitro data. Dosages necessary to achieve the MEC will depend on individual characteristics and route of administration. However, HPLC assays or bioassays can be used to determine plasma concentrations and the degree of inflammation in the subject.

Dosage intervals can also be determined using MEC value. Compositions of IbTI may be administered using a regimen which maintains plasma levels above the MEC for 10-90% of the time, preferably between 30-90% and most preferably between 50-90%. But in cases of local administration or selective uptake, the effective local concentration of IbTI may not be related to plasma concentration.

Many ailments and health problems can be ameliorated or even cured by adhering to a healthy diet. Chemical compounds and proteins in food and vegetables are increasingly being recognized as having a link to metabolic and physiological responses in the body. Food provides elements and compounds which are ingested, digested, absorbed, and circulated through the bloodstream to feed the cells of the body. Consumption of plant based foods provides a favorable source of essential nutrients. During the process of ingestion, digestion, and absorption, nutrients such as proteins, carbohydrates, and fats are broken down into their constituent molecules. Because of the variation of food digestion and absorption with each individual it is difficult to anticipate the absolute benefit for each person. Indeed, it is likely that some individuals will not experience the full benefits because of genetics, digestion patterns, weight, and also ingestion of chemicals that may interfere with the actions of food ingredients such as alcohol consumption and smoking. Thus, administration of drugs and other pharmacologically active substances requires purification, and addition of carriers to achieve a level high enough in the body to effect a positive change on the human body. One of the advantages of this invention is the purification and combination with a carrier that permits a pharmacologically effect dose to be administered to the patient. Treatment with IbTI and a pharmacological carrier may be superior in the treatment of inflammation and hyperalgesia than raw or unpurified sources of pharmacologically active substances from food groups.

IbTI compositions disclosed herein can be evaluated for efficacy and toxicity using known methods. For example, the toxicology of the composition may be established by determining in vitro toxicity towards a cell line, such as a mammalian, and preferably human, cell line. The results of such studies are often predictive of toxicity in animals, such as mammals, or more specifically, humans. Alternatively, the toxicity of particular compositions in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition caused by inflammation and experience of pain. When selecting a model to determine efficacy, the skilled artisan can be guided by the state of the art to choose an appropriate model, dose, and route of administration, and regime. Of course, human clinical trials can also be used to determine the efficacy of a compound in humans. Markers associated with inflammation and pain may be assessed to determine the efficacy of treatment. For example, blood may be drawn before, during, and after treatment to quantify these markers. Proteins indicative of inflammation include cytokines, chemokines, prostaglandins, fibrin, immunoglobulins and bradykinin. Efficacy of treatment may be achieved by measurement of these proteins as well as the number and cell type in the blood and at the site of injury.

“iNOS” refers to inducible nitric oxide synthase. iNOS is an enzyme that catalyzes the production of nitric oxide. “iNOS production” refers to the synthesis of the inducible nitric oxide synthase in the form of protein, RNA, or DNA. “Inhibits iNOS” refers to a reduction, amelioration or suppression of amount, concentration, or function of iNOS in the cells, tissue, or body of the subject. Inhibition of iNOS may also occur via the elimination of, or reduced stability of, iNOS in vivo.

“Inhibits NO production” refers to reduction, amelioration or suppression of the amount, concentration or change in function of nitric oxide.

The term “COX-2” refers to cyclooxygenase-2, a protein responsible for production of such molecules as prostanoids, prostaglandins, prostacyclin, and thomboxane. The term “inhibits COX-2 production” refers to the reduction, amelioration or suppression of amount, concentration, or change in function of iNOS in the cells, tissue, or body of the subject.

“TNF-α” refers to tumor necrosis factor-α. The term “inhibits TNF-αproduction” refers to a reduction, amelioration or suppression of amount, concentration, or change in function of TNF-α.

The term “inhibits PGE production” refers to reduction, amelioration or suppression of amount, concentration, or change in function of prostaglandin E2. The inhibition may occur in cells, tissues or organs of the subject.

The term “disorder” refers to a disease, physiological imbalance, infection, physical or psychological injury, or deviation from that which is considered a healthy homeostatic state.

“Edema” refers to a local or generalized condition in which body tissues contain an excessive amount of tissue fluid in the interstitial spaces. The edema may result from, for example, increased permeability of capillary walls, lymphatic obstruction, inflammatory conditions, presence of bacterial toxins, venoms, or histamine.

MDA production can be a result of free radical attack on plasma membranes. MDA detects the oxidative degradation of lipids in biological samples. MDA production can be linked to attack by free radicals upon the membranes of cells. “Inhibits MDA production” refers to a reduction in this oxidative stress and may be observed by a reduction in the amount, concentration, or distribution of degraded lipids and/or free radicals

Protein kinase C is a family of enzymes that modulate protein function through the phosphorylation of amino acids. “Inhibits protein kinase C (PKC) production” refers to the resulting anti-inflammatory or anti-hyperalgesic effect mediated by modulation of PKC activity. The modulation of PKC activity may effected by decreasing or increasing the amount, concentration, or activity of PKC. Modulation may also occur by changing the target of phosphorylation.

### EXAMPLES

LPS (endotoxin from Escherichia coli, serotype 0127:B8), and other chemicals were purchased from Sigma Chemical Co. (St. Louis, USA). TNF-α ELISA Assay Kit and Prostaglandin E₂ immunoassay kit (Assay Designs Inc., Ann Arbor, USA), anti-iNOS, anti-COX-2, anti-MMP-9, and anti-β-actin antibody (Santa Cruz, USA) and a protein assay kit (Bio-Rad Laboratories Ltd., Watford, Herts, U.K.) were obtained as indicated. Poly-(vinylidene fluoride) membrane (Immobilon-P) was obtained from Millipore Corp. (Bedford, Mass., USA).

Male ICR mice (18-25 g) were obtained from the BioLASCO Taiwan Co., Ltd. The animals were kept in plexiglass cages under a constant temperature of 22±1° C., relative humidity 55±5%, and a light regime of 12 hours dark/12 hour light for at least 2 weeks before the experiment. They were given food and water ad libitum. All experimental procedures were performed according to the NIH Guide for the Care and Use of Laboratory Animals. The placebo groups were given subcutaneous 0.1 ml/10 g saline using a bent blunted 27-gauge needle connected to a 1 ml syringe. All tests were conducted under the guidelines of the International Association for the Study of Pain [Zimmermann, M., Ethical guidelines for investigations of experimental pain in conscious animals. Pain. 1983, 16, 109-110].

Means of triplicate were calculated. Student's t test was used for comparison between two treatments. A difference was considered to be statistically significant when p<0.05, p<0.01 or p<0.001.

### Example 1A

### Effect of Sweet Potato Trypsin Inhibitor (IbTI) on Lipopolysaccharide (LPS)-Induced Cell Viability of Raw 264.7 Macrophages

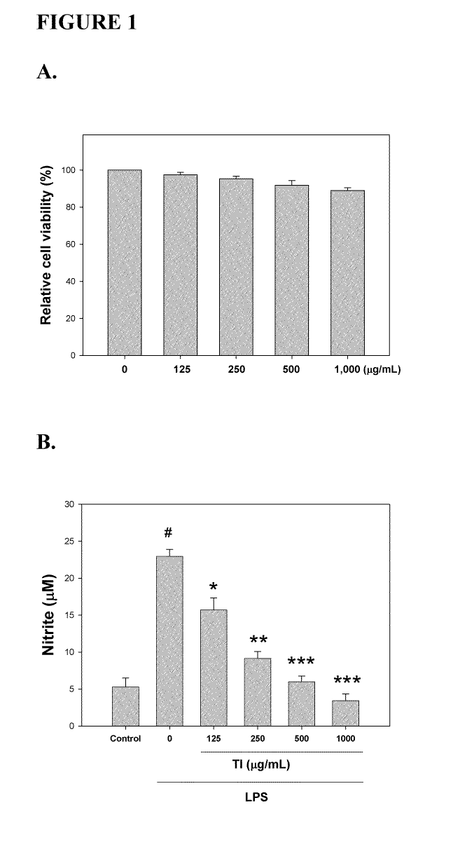

The murine macrophage cell line RAW 264.7 (BCRC No. 60001) was purchased from the Bioresources Collection and Research Center (BCRC) of the Food Industry Research and Development Institute (Hsinchu, Taiwan). Cells were cultured in plastic dishes containing Dulbecco's Modified Eagle Medium (DMEM, Sigma, St. Louis, Mo., USA) supplemented with 10% fetal bovine serum (FBS, Sigma, USA) in a CO2 incubator (5% CO2 in air) at 37° C. and subcultured every 3 days at a dilution of 1:5 using 0.05% trypsin-0.02% EDTA in Ca2+-, Mg2+-free phosphate-buffered saline (DPBS). For the viability assay, cells (2×105) were cultured in a 96-well plate containing DMEM supplemented with 10% FBS for 1 day to become nearly confluent. Then cells were cultured with 1 μg/mL LPS (lipopolysaccharide) for 24 hour in the absence or presence of IbTI (0, 5, 10, 20, 40 μM) (0, 125, 250, 500, 1,000 μg/mL). IbTI was added 1 hour before incubation with LPS. Cell viability measured using the MTT assay: cells were washed twice with DPBS and incubated with 100 μL of 0.5 mg/mL MTT for 2 hours at 37° C. testing for cell viability (MTT, (3-[4,5-dimethylthiazol-2-yl]-2,5-diphenyltetrazolium bromide) [Klebe, R. J. Rapid cloning of mammalian cells with honeycomb cloning plates and nonlethal vital stains. In Vitro. 1984, 20, 127-132]. The medium was then discarded and 100 μL dimethyl sulfoxide (DMSO) was added. After 30-min incubation, absorbance at 570 nm was read using a microplate reader. The result of iBTI treatment on cell viability can be seen in FIG. 1A.

### Example 1B

### Effect of Sweet Potato Trypsin Inhibitor (IbTI) on Lipopolysaccharide (LPS)-Induced NO Production of Raw 264.7 Macrophages

Cells were treated as above in Example 1A, and nitric oxide/nitrite levels in the cultured media and serum, which reflect intracellular nitric oxide synthase activity, were determined by Griess reaction. The cells were incubated with IbTI (0, 125, 250, 500, 1,000 μg/mL) in the presence of LPS (1 μg/mL) at 370 C for 24 h. Then, cells were dispensed into 96-well plates, and 100 mL of each supernatant was mixed with the same volume of Griess reagent (1% sulfanilamide, 0.1% naphthyl ethylenediamine dihydrochloride and 5% phosphoric acid) and incubated at room temperature for 10 min. Using sodium nitrite to generate a standard curve, the concentration of nitrite was measured by absorbance at 540 nm [Fiddler R. N. Collaborative study of modified AOAC method of analysis for nitrite in meat and meat products. J. Assoc. of Anal. Chem. 1977, 60, 594-599]. The data were presented as mean±S.D. for three different experiments performed in triplicate. # compared with sample of control group. *p<0.05 and **p<0.01 were compared with LPS-alone group. See FIG. 1B.

After treatment with LPS (1 μg/mL) for 24 h, the nitrite concentration increased in the medium. When RAW264.7 macrophages were treated with different concentrations of IbTI together with LPS (1 μg /mL) for 24 h, the IbTI inhibited nitrite production significantly (FIG. 1B). IbTI did not interfere with the reaction between nitrite and Griess reagents at 1,000 μg/mL. Unstimulated macrophages, after 24 hours of incubation in culture medium produced background levels of nitrite. When RAW 264.7 macrophages were treated with different concentrations of IbTI (0, 125, 250, 500, and 1000 μg/mL, corresponding to the 0, 5, 10, 20, and 40 μM respectively) together with LPS (1 μg/mL) for 24 h, a significant concentration-dependent inhibition of nitrite production was detected. There was either a significant decrease in the nitrite production of group treated with 125 μg/mL IbTI (p<0.05), or very or highly significant decrease of groups treated respectively with 250, 500 and 1,000 μg/mL of IbTI when compared with the LPS-alone group (p<0.01 or p<0.001).

### Example 2A & 2B

### Effects of Sweet Potato Trypsin Inhibitor on TNF-α and PGE2 Production by LPS-Induced Raw 264.7 Macrophages

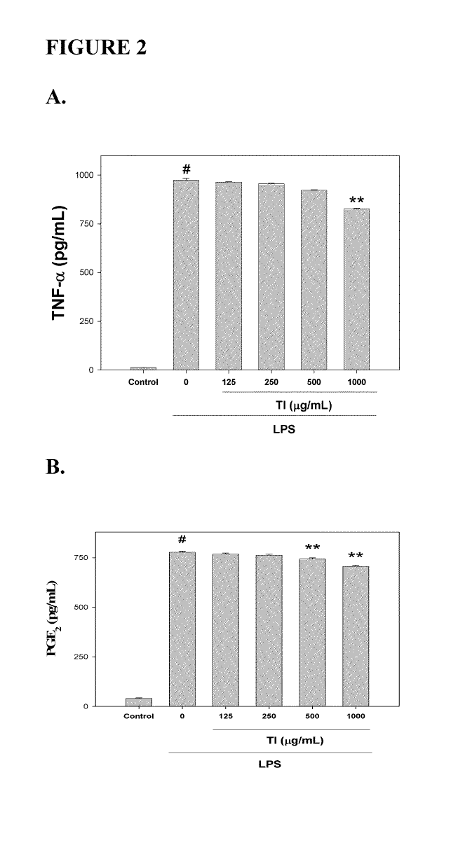

Cells (1×10⁶/well) were incubated with IbTI (0, 125, 250, 500, and 1,000 μg/mL) in the presence of LPS (1 μg/mL) at 37° C. for 24 h. Levels of TNF-α in cultured media and serum were determined using a commercial available enzyme linked immunosorbent assay (ELISA). The concentration of TNF-α was photometrically determined using a microplate reader (Molecular Devices, Sunnyvale, Calif., USA) at 405 nm (FIG. 2A).

TNF-α mediates the production of many other cytokines during inflammation, in particular the production of IL-1β and IL-6 [Locksley, R. M.; Killeen, N.; Lenardo, M. J. The TNF and TNF receptor superfamilies: integrating mammalian biology. Cell. 2001, 104, 487-501]. We examined the effect of IbTI on LPS induced up-regulation of TNF-α. A very low amount of TNF-α protein was detected by a specific ELISA for TNF-α in controls (FIG. 2A). Escherichia coli LPS (1 μg/mL) induced large quantities of TNF-α production, which was inhibited by pretreatment one hr before LPS with IbTI for 24 hr in a dose dependent manner, reaching 15% inhibition at the highest dose tested (1,000 μg/mL). Thus, IbTI very significantly blocks LPS-induced production of TNF-α protein (p<0.01).

PGE₂ was determined using a prostaglandin E₂ immunoassay kit. The concentration of PGE₂ was determined using a microplate reader (Molecular Devices, USA) at 405 nm. See FIGS. 2A & 2B. The data were presented as mean±S.D. for three different experiments performed in triplicate. # compared with sample of control group. **p<0.01 was compared with LPS-alone group.

An increase of PGE₂ production has been demonstrated by LPS treatment. Therefore, we investigated the effects of IbTI on LPS-induced PGE₂ production in macrophage. After treatment with LPS (1 μg/mL) for 24 h, the amount of PGE₂ elevated clearly in the medium, and IbTI at 500 or 1,000 μg/mL in the presence of LPS (1 μg/mL) was able to significantly suppress the LPS-induced production of PGE₂ in RAW 264.7 macrophages when compared with the LPS-alone group (p<0.01) (FIG. 2B).

### Example 3A & B

### Inhibition of iNOS and COX-2 Protein Expression by Sweet Potato Trypsin Inhibitor in LPS-Stimulated RAW264.7 Cells

Cells were incubated for 24 hours with 1 μg/ml of LPS in the absence or the presence of IbTI.

Cells were then collected by centrifugation at 700 g for 10 min, then the pellets were resuspended in a lysis buffer (40 mM Tris-HCl (pH 8.0), 1% NP-40, 1 mM phenylmethylsulfonyl fluoride (PMSF), 150 mM NaCl) at 4° C. for 15 min. Cell lysates (50 μg of proteins per lane) were fractionated on 12.5% SDS-polyacrylamide gels prior to being transferred to the membrane (Immobilon-P membranes, Millipore, Bedford, Mass., USA) according to that described by the manufacturer. Membranes were blocked for 2 hours at room temperature in 5% nonfat dry milk powder and then incubated with primary antibody. After incubation, membranes were washed in phosphate-buffer saline with 0.05% Tween (PBST) three times, 10 minutes each, then incubated with anti-rabbit alkaline phosphatase-conjugated antibody, washed in PBST three times, 10 minutes each, and developed using NBT (nitro blue tetrazolium)/BCIP (5-bromo-4-chloro-3-indolyl-phosphate) (Sigma, USA). The second antigen (goat against rabbit Fc portion of Ig) was a product of Sigma (USA). β-actin was used as an indicator to assure equality of lane loading. Blots were also stained with Coomassie Brilliant Blue to confirm that equal amounts of protein extract were present in each lane.

In order to investigate whether the inhibition of NO production was due to a decreased iNOS and COX-2 protein level, the effect of IbTI on iNOS and COX-2 protein expression was studied by immunoblot. Equal amounts of protein (50 μg/lane) were resolved by SDS-PAGE and then transferred to a nitrocellulose membrane and iNOS and COX-2 protein was detected using a specific antibody.

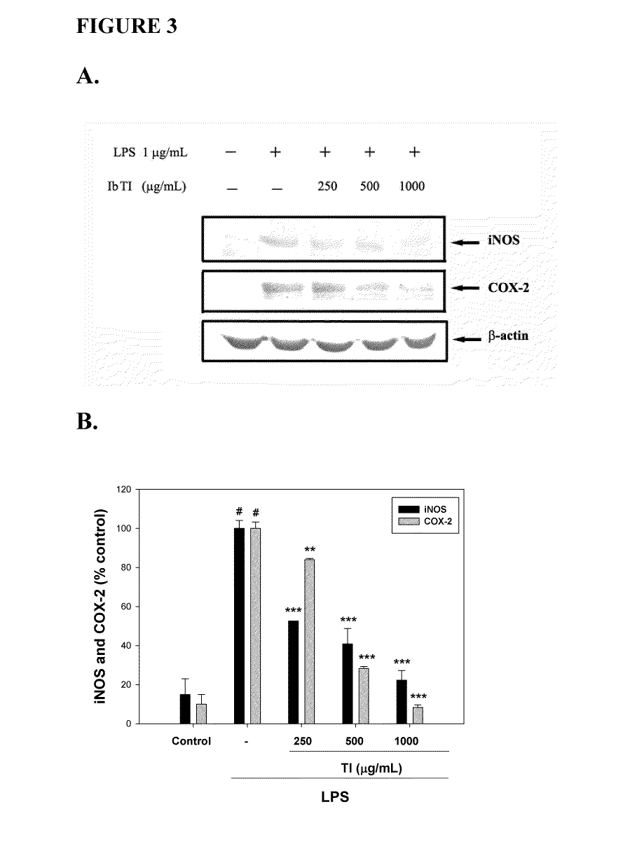

FIG. 3A is a representative western blot from two separate experiments. The results showed that incubation with IbTI (0, 250, 500, and 1,000 μg/mL) in the presence of LPS (1 μg/mL) for 24 hours inhibited iNOS protein expression in mouse macrophage RAW 264.7 cells in a dose-dependent manner (FIG. 3A). The detection of β-actin was also performed in the same blot as an internal control. The intensity of protein bands were analyzed using Kodak Quantity software (Molecular Imaging Software System, Kodak) in three independent experiments.

In FIG. 3B, the relative iNOS and COX-2 protein levels were calculated with reference to a LPS-stimulated culture. There was an average of 77.8% and 91.6% down-regulation of iNOS and COX-2 protein, respectively, after treatment with IbTI at 1,000 μg /mL compared with the LPS-alone (FIG. 3B).

The data were presented as mean±S.D. for three different experiments performed in triplicate. **p<0.01 and ***p<0.001 were compared with LPS-alone group.

### Example 4

### Inhibition of MMP-9 and MMP-2 Protein Expression by Sweet Potato Trypsin Inhibitor in LPS-Stimulated RAW264.7 Cells

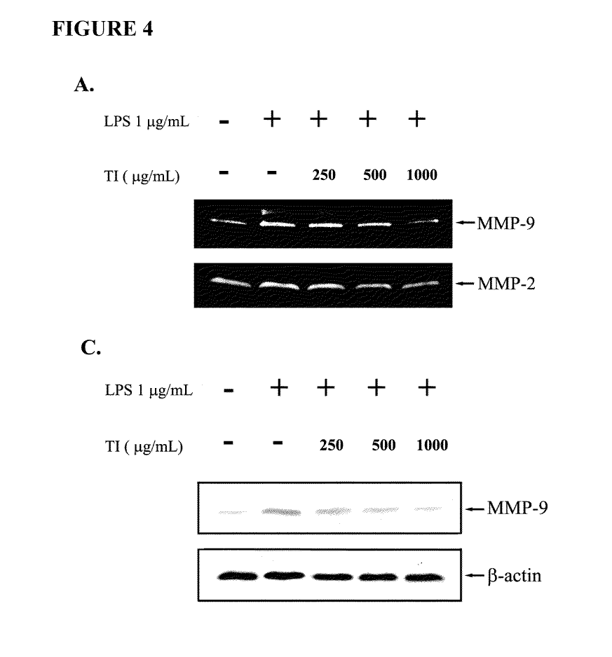

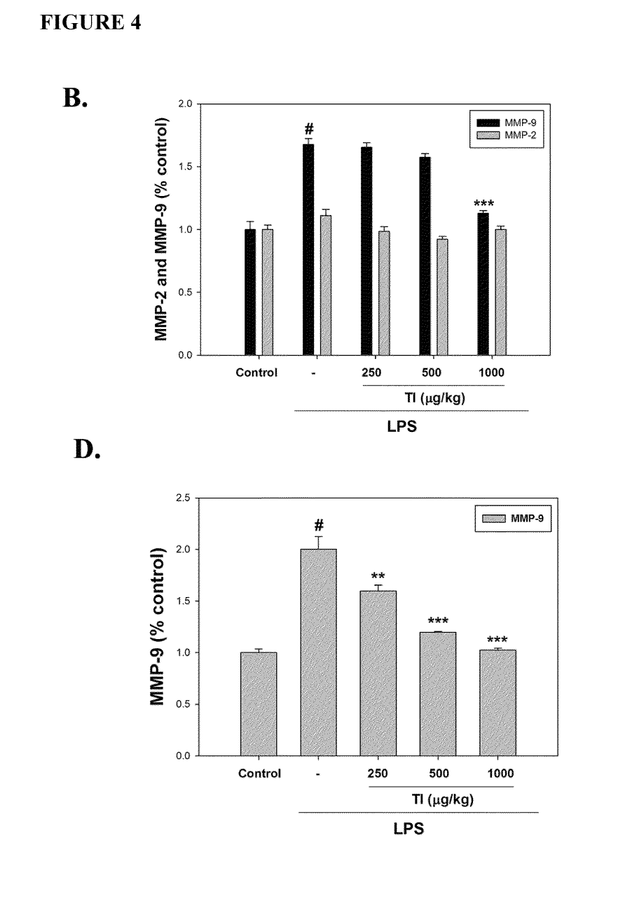

Cells were pretreated with IbTI (0, 10, 20, and 40 μM) (0, 250, 500, and 1,000 μg/mL) for 1 hour before being incubated with LPS (1 μg/mL) for 24 h. (A) Cells suspended were then prepared and the MMP-9 and MMP-2 activity were detected using SDS-PAGE zymography. (B) Relative MMP-9 and MMP-2 protein levels were calculated with reference to a LPS-stimulated control culture. (C) Inhibition of MMP-9 protein expression by IbTI in LPS-stimulated RAW264.7 cells using an antibody specific for MMP-9. β-actin was used as an internal control. (D) Relative MMP-9 protein level was calculated with reference to a LPS-stimulated control culture. The data were presented as mean±S.D. for three different experiments performed in triplicate. ***p<0.001 was compared with positive control group. See FIG. 4.

MMPs play an important role in the degradation and remodeling of the extracellular matrix at sites of inflammation. MMP-9 was greatly induced in LPS-stimulated RAW264.7 macrophage cells. Treatment with IbTI was able to suppress the induction of MMP-9 in the stimulated macrophages (FIG. 4A). IbTI at 1,000 μg/mL compared with the LPS-alone significantly decreased MMP-9 expression as shown by SDS-PAGE zymography (p<0.001) (FIG. 4B).

In FIG. 4C, the level of MMP-9 protein was analyzed by western blotting to determine the inhibitory mechanism of MMP-9 production by IbTI in LPS-stimulated RAW 264.7 cells. Expression of the MMP-9 protein was barely detectable in unstimulated cells, but markedly increased after LPS (1 μg/mL) stimulation (FIG. 4C). Treatment of LPS-stimulated macrophages with IbTI (0, 250, 500, and 1,000 μg/mL) caused dose-dependent inhibition of MMP-9 expression at the protein level in a dose-dependent manner. The detection of β-actin was also performed in the same blot as an internal control. The intensity of protein bands were analyzed using Kodak Quantity software in three independent experiments and showed an average of 40.1% and 48.9% down-regulation of MMP-9 protein expression, respectively, after treatment with IbTI at 500 and 1,000 μg/mL compared with the LPS-alone (FIG. 4D) (p<0.001).

### Example 5

### Effects of Sweet Potato Trypsin Inhibitor and Indomethacin (Indo) on Hind Paw Edema Induced by λ-Carrageenan in Mice

The anti-inflammatory activity of IbTI was determined by the Carr-induced edema test in the hind paws of mice as can be seen in FIG. 5. Male ICR mice (eight per group), 18 g to 25 g, were fasted for 24 hours before the experiment with free access to water. Fifty microliters of a 1% suspension of Carr in saline was prepared 30 minutes before each experiment and was injected into the plantar side of right hind paws of the mice. The IbTI or indomethacin was suspended in Tween-80 in 0.9% (w/v) saline solution. The final concentration of Tween-80 did not exceed 5% and did not cause any detectable inflammation. Two hours after Carr treatment, IbTI at the doses of 10, 20 and 40 mg/kg were administered intra-peritoneally. For a positive control, indomethacin was administered intra-peritoneally at a dose of 10 mg/kg before the Carr treatment [Mascolo et al., J. Ethnopharmacol. 1989, 27, 129-140]. Paw volume was measured immediately after Carr injection and at 1, 2, 3, 4 and 5 hours intervals after the administration of the edematogenic agent using a plethysmometer (model 7159, Ugo Basile, Varese, Italy). The degree of swelling induced was evaluated by the ratio a/b, where a is the volume of the right hind paw after Carr treatment, and b is the volume of the right hind paw before Carr treatment.

As shown in FIG. 5, Carr induced paw edema. IbTI (40 mg/kg) significantly prevented (p<0.05) the development of paw edema 5 hours after Carr stimulation. Indomethacin (10 mg/kg), as a positive control, significantly decreased paw edema 3, 4 and 5 hours after Carr injection (p<0.01 or p<0.001).

The Carr test is highly sensitive to nonsteroidal antiinflammatory drugs, and has long been accepted as a useful phlogistic model for investigating new drug therapies [Just et al., Planta Med. 1998, 64, 404-407]. The degree of swelling of the Carr-injected paws was maximal 4 hours after injection. Statistical analysis revealed that IbTI and indomethacin significantly inhibited the development of edema caused by Carr injection. It is well known that the edema-induced by Carr is characterized by the presence of prostaglandins and other compounds of slow reaction such as bradykinin which later induces the biosynthesis of prostaglandin and other autacoids, which are responsible for the formation of the inflammatory exudates [Spector, W. G. & Willoughb, D. A. Bacteriol. Rev. 1963, 27, 117-154; Ueno et al., Life Sci. 2000, 66, 155-160]. Besides, in the Carr-induced rat paw edema model, the production of prostanoids was shown to go through the serum expression of COX-2 by a positive feedback mechanism [Dudhgaonkar et al., Life Sci. 2006, 78, 1044-1058]. Therefore, one suggestion is that the action mechanism of IbTI may be related to prostaglandin synthesis inhibition, as described for the anti-inflammatory mechanism of indomethacin in the inhibition of the inflammatory process induced by Carr [Kirkova et al., General Pharmacology. 1992, 23, 503-507].

The data were presented as mean±S.D. for three different experiments performed in triplicate. **p<0.01 and ***p<0.001 were compared with the λ-carrageenan (Carr) group.

### Example 6

### Effects of Sweet Potato Trypsin Inhibitor and Indomethacin (Indo) on the Malondialdehyde Concentration of Mice Paws

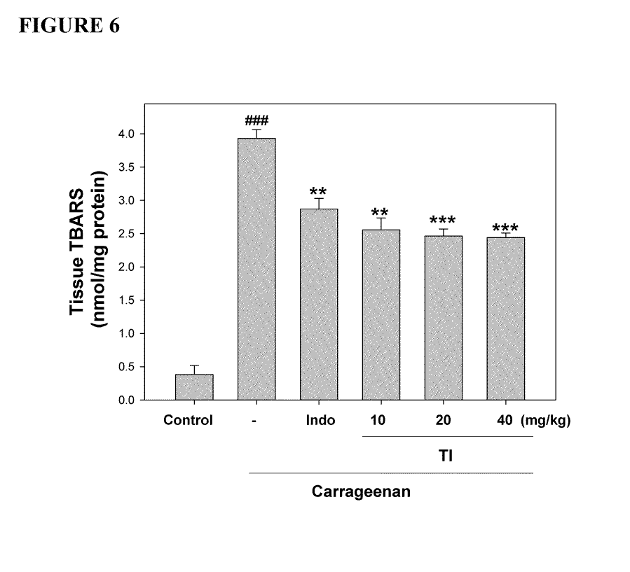

Malondialdehyde (MDA) was evaluated by the thiobarbituric acid reacting substance (TBARS) method; see FIG. 6 [Tatum et al., Lipids. 1990, 25, 226-229]. The principle is that MDA reacted with thiobarbituric acid at high temperature and formed a red-complex TBARS. The absorbance of TBARS was determined at 532 nm. Each value represents mean±S.D. ###p<0.001 as compared with the control group. **p<0.01 and ***p<0.001 were compared with the λ-carrageenan (Carr) group.

FIG. 6 shows that IbTI at 10 mg/kg decreased MDA level in the edema paw very significantly 5 hours after Carr injection (p<0.01); and IbTI at 20 or 40 mg/kg decreased MDA level in the edema paw highly significantly 5 hours after Carr injection (p<0.001).

Some papers demonstrate that inflammatory effect induced by Carr could be associated with free radicals. Free radicals, prostaglandin and NO will be released when administrating with Carr for 1-5 hours [Janero, D. R., Free Radic. Biol. Med. 1990, 9, 515-540]. The edema effect was raised to maximum at the third hour [Tonussi et al., Pain. 1999, 82, 81-87]. MDA production is due to free radical attack on plasma membrane [Salvemini et al., Eur. J. Pharmacol. 1996, 303, 217-220]. Thus, inflammatory effect would result in the accumulation of MDA.

### Example 7

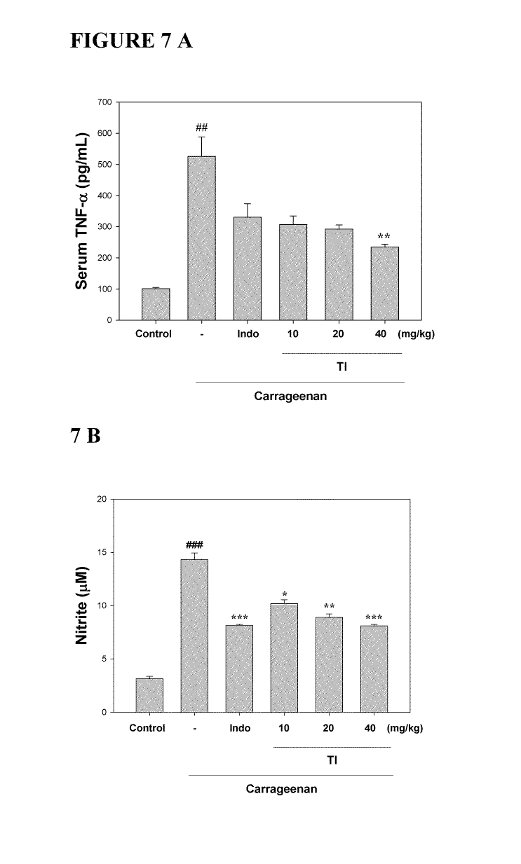

### Effects of IbTI and Indomethacin (Indo) on Carrageenan (Carr)-Induced TNF-α (FIG. 7A) and NO (FIG. 7B) Concentration of Serum at 5th Hour in Mice

The data were presented as mean±S.D. for three different experiments performed in triplicate. ###p<0.001 as compared with the control group. *p<0.05, **p<0.01 and ***p<0.001 were compared with the λ-carrageenan (Carr) group.

IbTI at 40 mg/kg decreased the TNF-α level in serum very significantly 5 hours after Carr injection (p<0.01) (FIG. 7A). IbTI (10, 20 and 40 mg/kg) decreased the NO level in serum 5 hours after Carr injection. IbTI at 10 mg/kg significantly decreased the serum NO level (p<0.05); and IbTI at 20 and 40 mg/kg, respectively, decreased the serum NO level very significantly and highly significantly (p<0.01 or p<0.001) (FIG. 7B).

TNF-α is a mediator of Carr-induced inflammation, and is able to induce the further release of kinins and leukotrienes, which is suggested to have an important role in the maintenance of long-lasting nociceptive response [Cuzzocrea et al., Life Sci. 1997, 60, 215-220]. In this study, we found IbTI decreased the TNF-α level in serum after Carr injection.

L-arginine-NO pathway has been proposed to play an important role in the Carr-induced inflammatory response [Cuzzocrea et al., Life Sci. 1997, 60, 215-220]. Our present results also confirm that Carr-induced paw edema results in the production of NO. The expression of the inducible isoform of NO synthase has been proposed as an important mediator of inflammation [Cuzzocrea et al., Life Sci. 1997, 60, 215-220].

### Example 8

### Histological Appearance of the Mouse Hind Footpad after a Subcutaneous Injection with 0.9% Saline (Control Group) or Carrageenan (Carr) Stained with Hematoxylin and Eosin

For histological examination, biopsies of paws were taken 5 hours after Carr injection into the plantar side of right hind paws. The tissue slices were fixed in solution with 1.85% formaldehyde and 1% acetic acid for 1 week at room temperature, then dehydrated by graded ethanol and embedded in Paraffin (Sherwood Medical, St. Louis, Mo., USA). Sections (thickness 5 μm) were deparaffinized with xylene and stained with hematoxylin and eosin. All samples were observed and photographed with BH2 Olympus microscopy (Japan). Every 3-5 tissue slices were randomly chosen from control, Carr, Indo and IbTI-treated (40 mg/kg) groups. The numbers of neutrophils were counted in each scope (400×) and average counts from 5 scopes of each tissue slice were calculated.

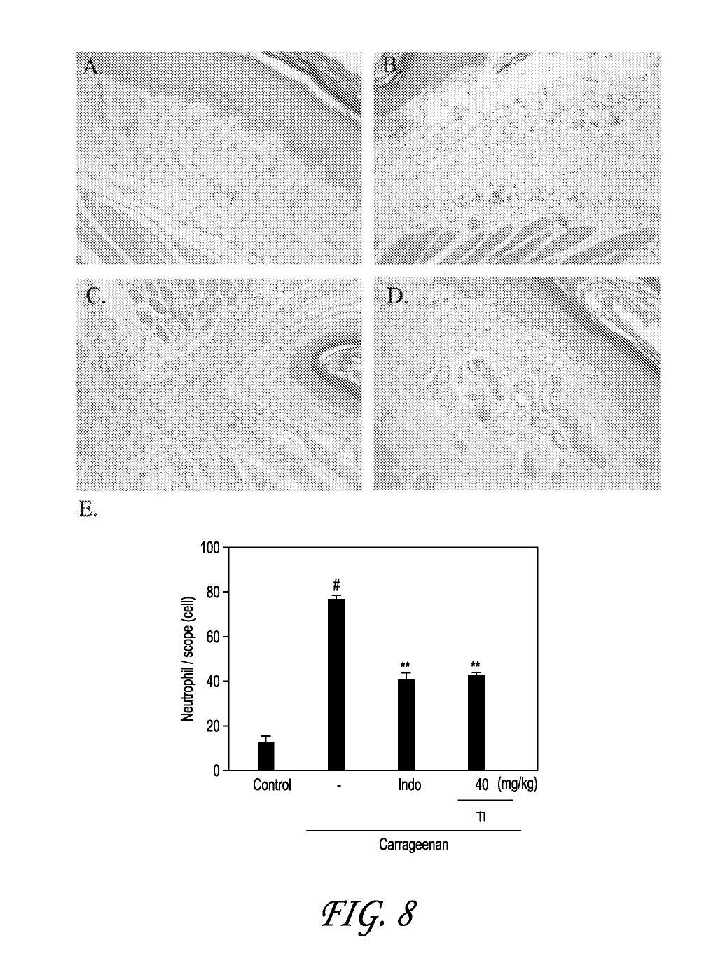

Control rats (FIG. 8A): show the normal appearance of dermis and subdermis without any significantly lesion. FIG. 8B shows hemorrhage with moderately extravascular red blood cell and large amount of inflammatory leukocyte mainly neutrophils infiltration in the subdermis interstitial tissue of mice following the subcutaneous injection of Carr only. Moreover, detail of the subdermis layer show enlargement of the interstitial space caused by edema with exudates fluid. FIG. 8C shows indomethacin (Indo) significantly reduced the level of hemorrhage, edema and inflammatory cell infiltration compared to subcutaneous injection of Carr only. IbTI (40 mg/kg) significantly shows morphological alterations compared to subcutaneous injection of Carr only (FIG. 8D). (100×.) The numbers of neutrophils were counted in each scope (400×) and their average counts from 5 scopes of every tissue slice were calculated (FIG. 8E). **P<0.01, compared with Carr group.

Paw biopsies of control mice showed marked cellular infiltration in the connective tissue. The infiltrates accumulated between collagen fibers and into intercellular spaces. Control groups showed the normal appearance of dermis and subdermis without any significantly lesion (FIG. 8A). Hemorrhage with moderately extra vascular red blood cell and large amount of inflammatory leucocytes mainly neutrophil infiltration in the subdermis interstitial tissue of mice following the subcutaneous injection of Carr only. Moreover, detail of the subdermis layer show enlargement of the interstitial space caused by edema with exudate fluid (FIG. 8B). The Carr plus IbTI (40 mg/kg) group showed a reduction in inflammatory response to Carr. Actually inflammatory cells were reduced in number and confined to near the vascular areas. Intercellular spaces did not show any cellular infiltrations. Collagen fibers were regular in shape and showed a reduction of intercellular spaces. Moreover the hypoderm connective tissue was not damaged. Following the subcutaneous injection of Carr combined with IbTI significant morphological alterations were observed compared to Carr only. The lesions show no hemorrhage and markly reduced the number of inflammatory neutrophil infiltration in the subdermis interstitial tissue (FIG. 8C). Moreover, no edema was seen in the interstitial space. (100×.) Neutrophils were found to increase with Carr treatment (P<0.001). Indomethacin (10 mg/kg), as a positive control, significantly decreased the number of inflammatory neutrophil infiltration (p<0.01) (FIG. 8D). IbTI, as indomethacin, could significantly decrease the neutrophils numbers as compared to the Carr-treated group (P<0.01) (FIG. 8E).

### Example 9

### Effects of Sweet Potato Trypsin Inhibitor (IbTI) and Indomethacin (Indo) on MMP-9 and MMP-2 Expression Induced by λ-Carrageenan in Mice

For SDS-PAGE zymography, culture media (20 μl/sample) were electrophoresed on 8% (w/v) SDS-PAGE gel impregnated with 1 mg/mL gelatin under a non-reducing condition. The proteins in the gel were renatured by incubation with 2.5% Triton X-100 at room temperature for 1 h. The gel was stained with a solution of 0.1% Coomassie Brilliant Blue R-250. In this assay, clear zones against the blue background indicate the presence of gelatinolytic activity [Birkedal-Hansen, H. & Taylor, R. E. Biochem. Biophys. Res. Commun. 1982, 107, 1173-1178].

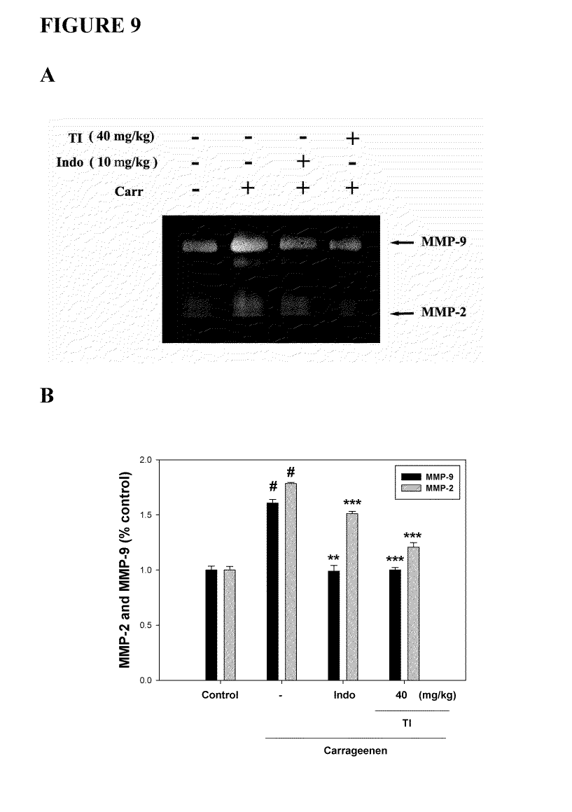

Tissue suspension was prepared and the MMP-9 and MMP-2 activity was detected using SDS-PAGE zymography (FIG. 9A). Relative MMP-9 and MMP-2 protein levels were calculated with reference to blank (FIG. 9B). The data were presented as mean±S.D. for three different experiments performed in triplicate. **p<0.01 and ***p<0.001 were compared with the λ-carrageenan (Carr) group.

The suppression of MMP-9 and MMP-2 expression treated with IbTI in Raw 264.7 macrophage cells. Cells were pretreated with IbTI (0, 10, 20, and 40 μM) (0, 250, 500, and 1,000 μg/mL) for 1 hour before being incubated with LPS (1 μg/mL) for 24 hours.

In this study IbTI at 40 mg/kg decreased the MMP-9 level in the paws significantly 5 hours after Carr injection (p<0.01) (FIG. 9A) as did indomethacin (10 mg/kg; p<0.01). Treatment with IbTI was able to suppress the induction of MMP-9 in the stimulated macrophages (FIG. 9B).

MMPs are a group of Zn-containing enzymes that degrade various components in extracellular matrix (ECM), including collagens, proteoglycans, gelatin, fibronectin, and glycoproteins [McCawley L. J. & Matrisian, L. M. Curr. Opin. Cell Biol. 2001, 13, 534-540]. MMPs influence the outcome of inflammatory reactions, angiogenesis, and cause the release of extracellular matrix-bound growth factors and cytokines that regulate many of these processes [Mott, J. D. & Werb, Z. Current Opinions in Cellular Biology. 2004, 16, 558-564]. MMP-9, the 92 kDa gelatinase B expressed in various cell lines such as keratinocytes, neutrophils and macrophages, are increased and activated in many kinds of inflammatory and malignant diseases such as periodontitis and pericoronitis. Its expression and activation is also induced by many inflammatory cytokines such as TNF-α [Sorsa et al., Annals of Medicine. 2006, 38, 306-321]. In the current study, IbTI was identified to reduce the induction of MMP-9 in the LPS-stimulated macrophages. Several anti-inflammatory agents were previously found to suppress the induction of MMP-9 in the stimulated cells.

### Example 10

### Extraction and Purification of Sweet Potato IbTI

Fresh storage roots of sweet potato (Ipomoea batatas (L.) Lam. ‘Tainong 57’) were purchased from a local market. Samples were washed, peeled, and then cut into strips that were extracted immediately [Huang et al., Plant Sci. 2005, 169, 423-431].

Extraction and purification of IbTI from sweet potato storage roots was carried out at 4° C. according to the method of Huang et al. [Huang et al., J. Agric. Food Chem. 2007, 55, 2548-2553]. The storage roots were cut into strips that were extracted immediately with four volumes (w/v) of 100 mM Tris-HCl buffer (pH 7.9) containing 100 mM NaCl, 1% (w/v) ascorbate and 1% (w/v) polyvinylpolypyrrolidone (PVPP) in a homogenizer for 30 s (four times). The homogenates were filtered through four layers of cheesecloth and centrifuged twice at 12,000×g for 30 min. The crude extracts were loaded directly onto a trypsin-Sepharose 4B affinity column (1.0×10 cm), and the adsorbed IbTI were eluted by changing pH value with 0.2 M KCl buffer (pH 2.0) [Huang, et al., J. Agric. Food Chem. 2007, 55, 6000-6006]. The extracts were desalted and concentrated with Centricon 10 and then lyophilized for further use. The SDS-PAGE analysis of purified IbTI showed a monomer with molecular mass of ca. 25 kDa. The yield was 8.3% (150 mg protein of purified TI/1,800 mg total protein of crude extract=8.3%).

The publications referred to herein are incorporated by reference in their entireties.

What is claimed is:

1. A pharmaceutical composition comprising an anti-inflammatory or an anti-hyperalgesic effective amount of isolated or purified Ipomomoea batatas trypsin inhibitor and a pharmaceutically acceptable carrier.

2. The composition of claim 1 wherein the Ipomomoea batatas trypsin inhibitor is derived from a sweet potato leaf or storage root.

3. A method of treating inflammation or hyperalgesia comprising
selecting a subject suffering from inflammation or hyperalgesia; and administering an anti-inflammatory or anti-hyperalgesia effective amount of the composition of claim 1 to the subject; wherein inflammation or hyperalgesia in the subject is reduced upon administration.

4. The method of claim 3, wherein the composition inhibits iNOS or COX-2 production.

5. The method of claim 3, wherein the composition inhibits TNF-α production.

6. The method of claim 3, wherein the composition inhibits PGE₂ production.

7. The method of claim 3, wherein the inflammation or hyperalgesia is associated with edema.

8. The method of claim 3, wherein the composition inhibits NO production.

9. The method of claim 3, wherein the composition inhibits protein kinase C (PKC) production.

10. A method of treating inflammation or hyperalgesia comprising
selecting a subject suffering from inflammation or hyperalgesia;
administering an anti-inflammatory or anti-hyperalgesia effective amount Ipomomoea batatas trypsin inhibitor;
wherein inflammation or hyperalgesia in the subject is reduced after the administering step.

11. The method of claim 10, wherein the iNOS or COX-2 production is inhibited in the subject after said administering step.

12. The method of claim 10, wherein TNF-α production is inhibited in the subject after said administering step.

13. The method of claim 10, wherein PGE₂ production is inhibited in the subject after said administering step.

14. The method of claim 10, wherein the inflammation or hyperalgesia is associated with edema.

15. The method of claim 10 wherein NO production is inhibited in the subject after said administering step.

16. The method of claim 10, wherein protein kinase C (PKC) production is inhibited in the subject after said administering step.

17. The method of claim 3, wherein the composition inhibits MDA production.

18. The method of claim 10, wherein MDA production is inhibited in the subject after said administering step.
